(19) United States
(12) Patent Application Publication
(10) Pub. No.: US 2024/0316329 A1
(43) Pub. Date: Sep. 26, 2024

(54) CONNECTION PROTECTOR, SECUREMENT SYSTEM HAVING THE CONNECTION PROTECTOR AND USES THEREOF

(71) Applicant: CHILDREN'S NATIONAL MEDICAL CENTER, Washington, DC (US)

(72) Inventors: Lori IRVIN, Washington, DC (US); Jules SHERMAN, Palo Alto, CA (US)

(73) Assignee: CHILDREN'S NATIONAL MEDICAL CENTER, Washington, DC (US)

(21) Appl. No.: 18/259,529

(22) PCT Filed: Dec. 23, 2021
(86) PCT No.: PCT/US2021/065100

§ 371 Date: Jun. 27, 2023

Related U.S. Application Data
(60) Provisional application No. 63/131,708, filed on Dec. 29, 2020.

(51) Int. Cl. A61M 39/10

(52) U.S. Cl. CPC A61M 39/1011; A61M 2039/1066; A61M 2205/0205

(57) ABSTRACT

Aspects of the disclosure provide a connection protector and a securement system including the connection protector. For example, the connection protector can include a first cover portion and a second cover portion, a combination of which forms an internal compartment configured to enclose a connection point of a first conveying device and a second conveying device. A hinge can be coupled to first sides of the first and second cover portions, enabling the first and second cover portions to be in open or close position. Two openings can be formed on both ends of the first and second cover portions, allowing the first and second conveying devices to pass therethrough, respectively. A first tamper-proof locking assembly attached to second sides of the first and second cover portions, the first tamper-proof locking assembly configured to lock the first cover portion to the second cover portion.

### INCORPORATION BY REFERENCE

The present disclosure claims the benefit of U.S. Provisional Application No. 63/131,708, “CONNECTION PROTECTOR AND USES THEREOF” filed on Dec. 29, 2020, which is incorporated herein by reference in its entirety.

### TECHNICAL FIELD

The present disclosure relates to the fields of connection protectors, tube protectors, tube holders, securement devices, or securement system for protecting the connection points or junctures of fluid and air conveying devices, such as tubes, drains, medical tubes or medical lines.

### BACKGROUND

There are many unexpected medical events which could occur during medical treatments when patients are connected to medical lines. Some unsupervised or bored patients may pull out or disconnect the medical lines which are physically attached to their bodies directly or indirectly. Other times, medical lines and tubes may accidently disconnect when patients are being transported or adjusting their position in their hospital room or bed. These unexpected events can sometimes go unnoticed and pose serious risks for injury and infection.

When a patient is hospitalized, clinicians connect external lines from the patient to a pump in order to administer therapeutics, such as fluids, medications, or nutrition, either intravenously or externally. For example, the external line can be a central line (a central venous catheter or a tube) which is placed in a large vein in the neck, chest, groin, arm or leg to give fluids, blood, or medications. The patient may have a peripheral an intravenous (IV) catheter connected to a line, a nasogastric (NG) tube, an ostomy tube, or various drainage tubes connected to a pump or drainage collection system. These lines, tubes, or drains can be connected easily and efficiently, but also can be easily disconnected. Various tube holders and securement devices are available to secure medical tubes and lines to prevent the disconnection. However, the performances of these products are not optimal. Securing and/or protecting the medical tubes or medical lines remains an unmet medical need. The present disclosure addresses these needs and provides products, devices or systems that have not been previously disclosed in the art.

### SUMMARY

The present disclosure provides a medical connection protector, a tube protector, a tube holder, a securement device or a securement system for protecting a connection point of a variety of tubes, drains, medical tubes, medical lines or connection points. In a preferred embodiment, the present disclosure provides a securement system including a tamper-proof or tamper-resistant connection protector to protect connection points of medical tubes or medical lines. The medical connection protector or securement system of the present disclosure provides protections to prevent accidental and unnoticed disconnection of life-saving lines and tubes which are attached to patients' bodies directly or indirectly.

In one embodiment, the present disclosure provides a medical connection protector including a first cover portion, a second cover portion, a hinge, two openings, at least one tamper-proof locking assembly and a clear visual access, wherein a combination of the first cover portion and the second cover portion forms an internal compartment of the medical connection protector. In one aspect, the internal compartment of the medical connection protector is configured to enclose a connection point of tubes. In one aspect, the process of unlocking the tamper-proof locking assembly is non-intuitive. In one aspect, the medical connection protector has color-coding for safety considerations. In one embodiment, the present disclosure provides a securement system including the medical connection protector and at least one securing patch, wherein the at least one securing patch is located adjacent to the opening of the medical connection protector.

Aspects of the present disclosure provide a medical connection protector for protecting a connection point of a first conveying device and a second conveying device. For example, the medical connection protector can include a first cover portion and a second cover portion, a combination of which forms an internal compartment configured to enclose a connection point of a first conveying device and a second conveying device. A hinge can be coupled to a first side of each of the first and second cover portions, the hinge configured to enable the first cover portion and the second cover portion to be in open or close position. Two openings can be formed on both ends of the first and second cover portions, allowing the first and second conveying devices to pass therethrough, respectively. A first tamper-proof locking assembly can be attached to a second side of each of the first and second cover portions, the first tamper-proof locking assembly configured to lock the first cover portion to the second cover portion.

For example, the first tamper-proof locking assembly can include a first female component attached to the second side of the first cover portion and a first male component attached to the second side of the second cover portion, the first female component and the first male component allowed to snap together in locked position. As another example, the first tamper-proof locking assembly can have a safety-lock mechanism, an engageable locking mechanism, or a releasable locking mechanism. In an embodiment, the medical connection protector further includes a second tamper-proof locking assembly that includes a second female component attached to the second side of the first cover portion and a second male component attached to the second side of the second cover portion, the second female component and the second male component allowed to snap together in locked position. In another embodiment, the medical connection protector of claim further includes a second tamper-proof locking assembly that includes a second female component attached to the second side of the second cover portion and a second male component attached to the second side of the first cover portion, the second female component and the second male component allowed to snap together in locked position.

For example, the hinge can be a living hinge integrated with the first sides of the first and second cover portions. As another example, the hinge is a mechanical hinge attached to the first sides of the first and second cover portions.

In an embodiment, one of the openings has the same diameter as an outer diameter of the first conveying device. In another embodiment, the medical connection protector can further include color-coding provided on the first cover portion.

In an embodiment, the medical connection protector can further include an inner core received in the internal compartment, the inner core configured to accommodate the connection point of the first and second conveying devices. For example, the inner core is a rigid inner core. In an embodiment, the inner core can have the same configuration of a shape of the connection point of the first and second conveying devices. In another embodiment, the inner core can have a rough surface. In some other embodiments, the medical connection protector can further include a monitoring system configured to monitor a state of the tamper-proof locking assembly and/or the first and second cover portions and transmit a signal based on the state.

In an embodiment, the medical connection protector can further include a fence closure disposed in the internal compartment, the fence closure configured to enclose the first conveying device and prevent the first conveying device from disconnection with the second conveying device. For example, the fence closure can include two walls installed on the first and second cover portions, respectively, the two walls overlapping when the first and second cover portions is in the close position.

In an embodiment, the medical connection protector can further include a visual access formed on the first cover portion, allowing the connection point of the first and second conveying devices to be visually accessed externally.

For example, the first cover portion can be made of a transparent material. As another example, the medical connection protector can be made of a biocompatible and/or antimicrobial material.

Aspects of the present disclosure further provide a securement system including the medical connection protector and at least one securing patch, the securing patch configured to secure the medical connection protector and/or the first conveying device to an object. For example, the securing patch can be a grip lock or a stat lock.

Note that this summary section does not specify every embodiment and/or incrementally novel aspect of the present disclosure or claimed disclosure. Instead, this summary only provides a preliminary discussion of different embodiments and corresponding points of novelty over conventional techniques. For additional details and/or possible perspectives of the present disclosure and embodiments, the reader is directed to the Detailed Description section and corresponding figures of the present disclosure as further discussed below.

### BRIEF DESCRIPTION OF THE DRAWINGS

Further features of the inventive concept, its nature and various advantages will be more apparent from the following detailed description, taken in conjunction with the accompanying figures:

FIG. 1 shows a diagram of a connection protector enclosing a connection point of two tubes according to an exemplary embodiment of the present disclosure;

FIG. 2 shows a configuration of a connection protector in open position enclosing a connection point of two tubes according to an exemplary embodiment of the present disclosure;

FIG. 3A shows a configuration of a connection protector in closed position in perspective view according to an exemplary embodiment of the present disclosure;

FIG. 3B shows a configuration of a connection protector in closed position in front view according to an exemplary embodiment of the present disclosure;

FIG. 3C shows a configuration of a connection protector in open position according to an exemplary embodiment of the present disclosure;

FIG. 4A shows a configuration of a connection protector in closed position enclosing a connection point of two tubes in perspective view according to an exemplary embodiment of the present disclosure;

FIG. 4A′ shows another configuration of a connection protector in closed position enclosing a connection point of two tubes in perspective view according to an exemplary embodiment of the present disclosure;

FIG. 4B shows a configuration of a connection protector in closed position enclosing a connection point of two tubes in front view according to an exemplary embodiment of the present disclosure;

FIG. 4C shows a configuration of a connection protector in open position enclosing a connection point of two tubes according to an exemplary embodiment of the present disclosure;

FIG. 5A shows a connection protector in closed position enclosing a connection point of two tubes in perspective and side views showing a double latch system and an opening of an internal compartment of the connection protector, respectively, according to an exemplary embodiment of the present disclosure;

FIG. 5B shows a connection protector in closed position enclosing a connection point of two tubes in perspective view showing a mechanical hinge according to an exemplary embodiment of the present disclosure;

FIG. 5C shows a connection protector in open position enclosing a connection point of two tubes according to an exemplary embodiment of the present disclosure;

FIG. 6 shows a diagram of a securement system including a connection protector and two securing patches for protecting a connection point of two tubes according to an exemplary embodiment of the present disclosure;

FIG. 7A shows the use of a securement system in combination of two dressings, wherein the securement system comprises a connection protector and two securing patches for protecting a connection point of two tubes according to an exemplary embodiment of the present disclosure;

FIGS. 7B and 7C show the use of multiple connection protectors and/or securement systems to secure medical lines which are attached directly or indirectly to the body of a patient according to an exemplary embodiment of the present disclosure;

FIG. 8 shows a securement system including a connection protector and two securing patches for securing and protecting a connection point of a drainage system including a drainage bag, a collection tube, and a Gastrostomy-tube (G-tube) extension according to an exemplary embodiment of the present disclosure,

FIG. 9 shows a securement system including a connection protector and two securing patches for securing and protecting a connection point of an extension feeding tubing and a kangaroo feeding set according to an exemplary embodiment of the present disclosure.

FIG. 10A shows a configuration of a connection protector in open position in top view according to an exemplary embodiment of the present disclosure;

FIG. 10B shows a configuration of a connection protector in open position in side view according to an exemplary embodiment of the present disclosure;

FIG. 10C shows a configuration of a connection protector in open position in front view according to an exemplary embodiment of the present disclosure;

FIG. 11A shows a configuration of a connection protector in open position in top view according to an exemplary embodiment of the present disclosure;

FIG. 11B shows a configuration of a connection protector in open position in side view according to an exemplary embodiment of the present disclosure; and

FIG. 11C shows a configuration of a connection protector in open position in front view according to an exemplary embodiment of the present disclosure.

### DETAILED DESCRIPTION

Throughout this description, the preferred embodiments and examples provided herein should be considered as exemplar, rather than as limitations of the present disclosure.

When patients are alone, bored, unsupervised or disoriented, they often play and pull on the medial lines that are attached directly or indirectly to their bodies. In particular, pediatric patients, and adult patients who experience altered mental status, have higher tendency of playing or pulling the medical lines when they are left alone or unattended. This is especially dangerous when they have central lines and other tubes or drains that become disconnected, as interruption of IV infusions or drainage of excess fluid from patient's bodies can lead to serious side effects, health risks and complications or death. Adult patients having any mental illness or disease process that causes confusion or irritability could include, but are not limited to dementia, Alzheimer's, Parkinson's Disease, alcohol/drug withdrawal, intensive care delirium, subacute infective psychosis, schizophrenia, stress, anxiety disorders, seizures or side effects from medications. Pediatric patients having any mental illness or disease process that causes confusion or irritability could include intensive care delirium, stress, anxiety disorders, seizures, drug related side effects, common neurological conditions or side effects from treatments related to a patient's diagnosis.

Lack of functional connection tubing may lead to hemodynamic instability, dehydration, nutrition deficits, and electrolyte imbalances in the body which may lead to severe physiologic and metabolic changes, or infections such as central line associated blood stream infections (CLABSIs), or catheter associated urinary tract infections (CAUTIs). When IV medication lines become disconnected and leak fluid, an unknown amount of medication is wasted. This makes it difficult for the clinician to determine the amount of medication delivered to the patient. In the case of antibiotics, it could lead to the interruption of effectively treating infections. The present disclosure provides a device and/or a system as a proactive approach to rectifying the problem of having disconnected medical lines, tubes and drains. This device and/or system can be applied to various patients including adult or pediatric patients.

The present disclosure provides a connection protector, a tube protector, a tube holder, a tube securement device or a tube securement system for protecting a connection point or a juncture of fluid and air conveying devices, such as tubes, drains, medical tubes or medical lines. The connection protector can be applied for use with a variety of IV infusion connection systems, such as IV infusion sets (BD Health, Medline, etc.), peripherally inserted central catheter (PICC) lines, central venous catheters (CVLs), Broviac Lines, Hickman Ports, Leonard Ports, Groshong Central Venous Catheters, Medport connections, Primary IV tubing, Secondary IV tubing, Double connector-Bifuse extension, Triple connector-Trifuse extension, Large bore connectors, Small bore connectors, Syringe pump tubing, Microclaves, and Y-type injection sites.

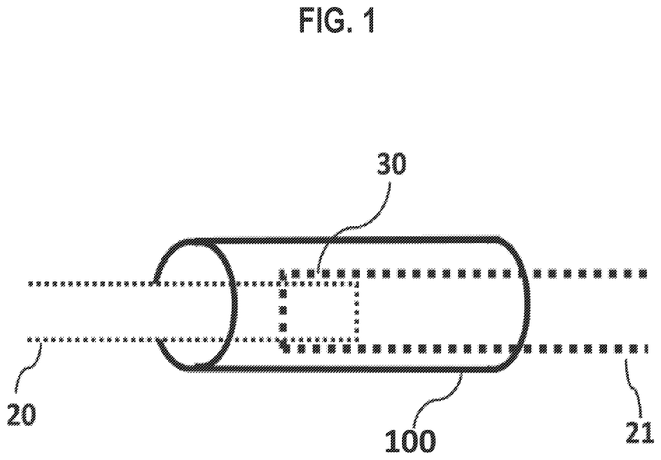

An exemplary embodiment of a connection protector 100 for enclosing a connection point of two conveying devices, such as tubes, lines and drains, is shown in FIG. 1. A first conveying device 20 and a second conveying device 21 are connected through a connection point (or a juncture) 30. The first conveying device 20 and the second conveying device 21 can be used to convey air, fluids, blood, or medication and include tubes, lines and/or drains. The first conveying device 20 and the second conveying device 21 are referred hereinafter as “the first tube 20” and “the second tube 21,” respectively for brevity. The connection protector 100 is used to enclose the connection point 30 of the first tube 20 and the tube 21.

In an embodiment, the connection protector 100 can be made of a biocompatible material. For example, the connection protector 100 can be made of medical-grade silicone, a ceramic biomaterial, such as alumina, bioglass and hydroxyapatite, metal, such as cobalt-chromium (Co—Cr) alloy, titanium (Ti), Ti alloy and stainless steel, or a polymer, such as polyvinylchloride (PVC), polyethylene (PE), polypropylene (PP), polytetrafluoroethylene (PTFE), polymethyl methacrylate (PMMA), trimethylcarbonate and trimethylene carbonate (TMC) NAD-lactide. In another embodiment, the connection protector 100 can be made of plastic, such as antimicrobial plastics (i.e., synthetic polymer materials that contain integrated and active anti-microbial additives) that are resistant to microorganisms, to prevent CLABSIs or CAUTIs.

In one embodiment, the first tube 20 and the second tube 21 can include tunneled central venous catheters (CVCs), non-tunneled CVSs and/or PICC lines, which can be made of polyurethane (PU), polyethylene terephthalate (PET) or silicone materials. In another embodiment, the first tube 20 and the second tube 21 can include drains, drainage tubes, medical tubes, medical lines, medical central lines, medical peripheral lines, medical Christmas tree connectors, luer locks, ports, IV tubes, NG tubes, ostomy tubes, gastrostomy tube (G-tube) connections, orogastric tubes (OG tubes), G-tube extensions, gastrojejunostomy tube (GJ-tube) connections, feeding tube lines, feeding bag lines, feeding extension tubings, special ostomy drains, duodenostomy drains, Jackson Pratt drains (JP drains), chest tube drains, chest tube connections, wound vac connections, urine catheter connections, rectal tube connections, rectal tube drains, lumbar drains, and/or cerebrospinal fluid drains from the skull and spine. For example, Becton, Dickinson and Company (BD) offers a variety of tubing sets, including Gravity administration sets (see bd.com/en-us/offerings/capabilities/infusion-therapy/iv-administration-sets/iv-gravity-and-secondary-sets/gravity-administration-sets) and IV administration sets (see bd.com/en-us/offerings/capabilities/infusion-therapy/iv-administration-sets). As another example, Qosina offers a plurality of parts of extension lines, such as part #33067 (0.965 ID×2.21 mm OD), part #33066 (0.5 ID×2 mm OD) and part #33068 (1.27 ID×2.2 mm OD), all of which feature a female luer lock connector on one tube end and a male luer lock connector on the other (see qosina.com/extension-lines-2).

The first tube 20 and the second tube 21 can be of the same or different sizes. For example, the first tube 20 and the second tube 21 can include IV infusion sets with diameters of 0.55-1.5 mm, 3 mm or 4 mm, primary IV tubing with diameters similar to the IV infusion sets, secondary IV tubing with diameters similar to the IV infusion sets, Double connector-Bifuse extension with diameters similar to the IV infusion sets, Triple connector-Trifuse extension with diameters similar to the IV infusion sets, PICC lines with diameters of 1.42 mm, 1.70 mm, 1.73 mm, 1.98 mm, 2 mm, 2.06 mm, 2.5 mm, 3 mm, 3.5 mm, 4 mm, 4.5 mm, 5 mm, 5.5 mm, 6 mm, 7 mm or 8 mm, CVCs with diameters of 1.8 mm, 2.4 mm or 2.5 mm, Large bore connectors with diameters of 6 mm, 6.8 mm, 7.1 mm or 8 mm, Broviac Lines with diameters of 0.95 mm, 1.45 mm or 2.2 mm, Hickman Ports with diameters of 0.7 mm, 0.8 mm, 1.0 mm, 1.3 mm, 1.5 mm, 1.6 mm, 2.1 mm, 2.3 mm, 2.9 mm, 3.1 mm or 4.0 mm, Leonard Ports with diameters of 1.3 mm or 3.3 mm, Groshong Central Venous Catheters with diameters of 7 F (2.33 mm), 8 F (2.67 mm) or 9.5 F (3.167 mm), and/or G-Tubes with diameters of 4 F (1.33 mm), 5 F (1.67 mm), 6 F (2 mm), 7 F (2.33 mm), 8 F (2.67 mm), 9 F (3 mm), 10 F (3.33 mm), 12 F (4 mm), 14 F (4.67 mm), 16 F (5.33 mm), 18 F (6 mm) or 22 F (7.33 mm).

The first tube 20 and the second tube 21 may connect to a pump or a drainage collection system. For some patients, a central line (e.g., a central venous catheter) is placed in a large vein in the neck, chest, groin, leg or arm to give fluids, blood or medications or remove air and fluid from the chest. The central lines are the highest risk lines for infection risk if they become disconnected.

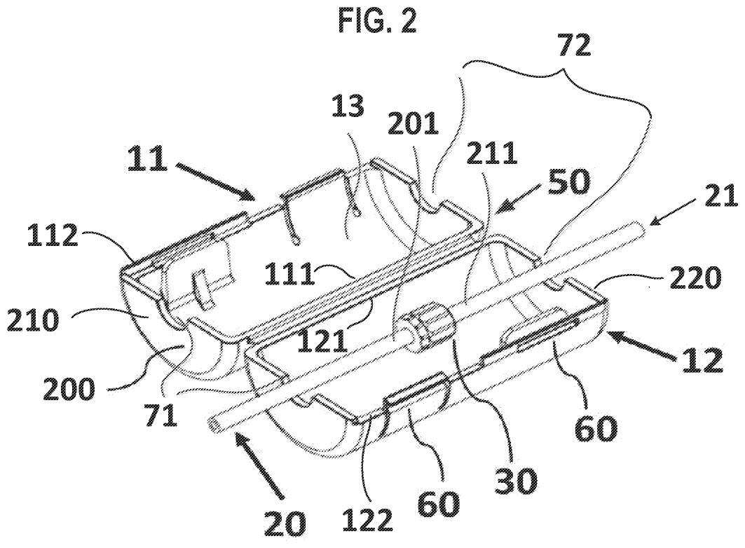

FIG. 2 is a schematic diagram of a connection protector 200 for enclosing a connection point of two conveying devices, such as tubes, lines and drains, according to some embodiments of the present disclosure. In one aspect, the connection protector 200 can include a first cover portion 11 and a second cover portion 12, a combination of which can form an internal compartment 13 of the connection protector 200 to enclose the connection point 30 of the first tube 20 and the second tube 21 and to accommodate an outlet 201 of the first tube 20 and an outlet 211 of the second tube 21.

The connection protector 200 can further include a hinge 50 connected to a first side 111 of the first cover portion 11 and a first side 121 the second cover portion 12 to allow opening and closing of the connection protector 200. In an embodiment, the hinge 50 can include a mechanical hinge, such as a pin hinge, attached to the first side 111 of the first cover portion 11 and the first side 121 of the second cover portion 21. In another embodiment, the hinge 50 can include a living hinge integrated with the first sides 111 and 121 of the first and second cover portions 20 and 21. Thus, the living binge 50 can provide a way to make or manufacture the first cover portion 11 and the second cover portion 12 of the connection protector 200 in one piece for ease of production. The living hinge 50 can also provide an advantage of reducing issues with mechanical failure at the hinge site. The living hinge 50 is a thin flexible hinge (flexure bearing) made from the same material connecting the two rigid pieces. The living hinge 50 is typically thinned or cut to allow the rigid pieces to bend along the axis line of the living hinge. The living hinge 50 ensures a single, compact design and allows for multiple opening/closing without loss of parts.

The connection protector 200 can further include at least one tamper-proof locking assembly 60 connected to a second side 112 of the first cover portion 11 and a second side 122 of the second cover portion 12. In one aspect, the tamper-proof locking assembly 60 of the connection protector 200 can have a tamper-proof locking mechanism, wherein the process of unlocking the tamper-proof locking mechanism is non-intuitive and requires a requisite force and dexterity, such as applying two hands with pressure for unlocking. In one aspect, the tamper-proof locking assembly 60 of the connection protector 200 can be a child-proofing, child-resistant, or baby-proofing safety lock. In one aspect, the tamper-proof locking mechanism can be easily unlocked by trained medical staff, but cannot be intuitively unlocked by hospitalized patients, including adult or pediatric patients. In one aspect, the connection protector 200 of the present disclosure is tamper-proof for protecting the connections of medical lines, tubes or drains which are attached directly or indirectly to the body of the patients. The tamper-proof feature of the connection protector 200 can disable the patient's ability to unscrew, pull apart, access or tamper with a line, tube, drain, or other medical line. In one aspect, the tamper-proof locking assembly 60 of the connection protector 200 is reusable for multiple uses, which can be open and closed repetitively. In one embodiment, the tamper-proof locking assembly 60 can include a double latch system, and the unlocking mechanism of the double latch system is non-intuitive and requires the action of two hands simultaneously. For example, the double latch system can include two latches and can be unlocked by pushing up the two latches in the same direction or in the opposite directions simultaneously. The double latch system can have a safety-lock mechanism, an engageable locking mechanism, or a releasable locking mechanism.

A proximal opening 71 and a distal opening 72 (when the connection protector 200 is in closed position) are located at a proximal end 210 and a distal end 220 of the connection protector 200, respectively, allowing the first tube 20 and the second tube 21 to pass therethrough. For example, the proximal and distal openings 71 and 72 can have the same diameters as the outer diameters of the first tube 20 and the second tube 21, respectively.

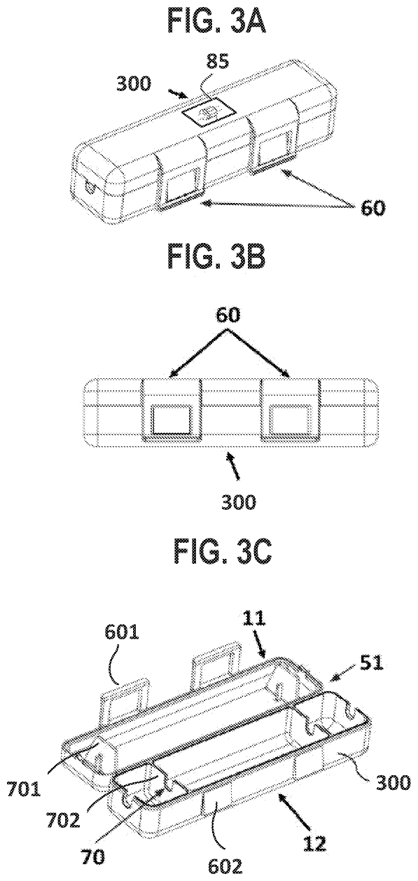

An exemplary embodiment of a connection protector 300 for enclosing a connection point of two conveying devices, such as tubes, lines and drains, is shown in FIG. 3A (perspective view), FIG. 3B (front view) and FIG. 3C (open position). The connection protector 300 also includes the first cover portion 11 and the second cover portion 12. A living hinge 51 connects the first cover portion 11 and the second cover portion 12. The connection protector 300 includes two of the tamper-proof locking assembly 60, such as a double latch system. In one exemplary embodiment, each of the tamper-proof locking assemblies (or the double latch system) 60 in FIGS. 3A-3C includes an open locking (female) component 601 in the first cover portion 11 and a protruding (male) component 602 in the second cover portion 12. In another embodiment, the open locking component 601 and the protruding component 602 of one of the tamper-proof locking assemblies 60 can be in the first cover portion 11 and the second cover portion 12, respectively, and the open locking component 601 and the protruding component 602 of the other of the tamper-proof locking assemblies 60 can be in the second cover portion 12 and the first cover portion 11, respectively. The design of the tamper-proof locking assembly 60 allows the two components 601 and 602 to snap together in locked position. It may require two hands to unlock the double latch system 60 with dexterity and precision. The double latch system 60 provides increased barriers to prevent intentionally or unintentionally disconnecting a connection point of a two tubes, e.g., the connection point 30 of the first tube 20 and the second tube 21 shown in FIG. 2, since it is required to add a requisite force, dexterity and/or cognitive ability to unlock the connection protector 300.

In one aspect, the connection protector 300 can further include two fence closures 70 with strain relief, as shown in FIG. 3C. The fence closure 70 with strain relief includes two walls 701 and 702 that enclose the tubing, e.g., the first tube 20 and the second tube 21 shown in FIG. 2, in the first cover portion 11 and the second cover portion 12 of the connection protector 300. The fence closure 70 with strain relief provides security from disconnection when there is stress or force on the first and second tubes 20 and 21.

In one aspect, the connection protector 300 can provide a clear visual access 85 or a clear visual indication of the connection point 30 of the first and second tubes 20 and 21 for safety considerations, as shown in FIG. 3A. In one aspect, the visual access 85 of the connection protector 300 can be made of clear or transparent material. In another aspect, the connection protector 300 (or the first and second cover portions 11 and 12) can be made of clear or transparent material. The clear or transparent appearance of the connection protector 300 provides the advantage of allowing the medical staff to quickly verify the connection point 30 without opening the connection protector.

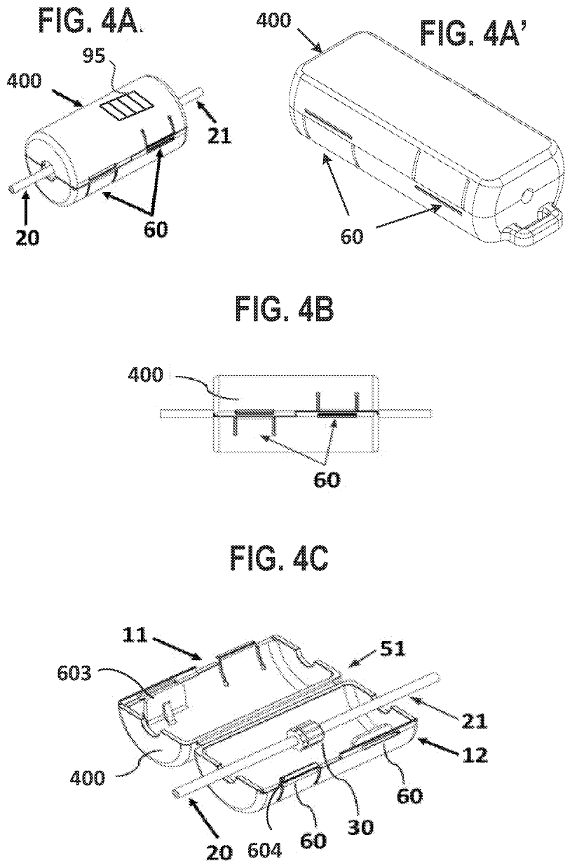

An exemplary embodiment of a connection protector 400 for enclosing a connection point of two conveying devices, e.g., tubes, lines and drains, is shown in FIG. 4A (perspective view), FIG. 4B (front view) and FIG. 4C (open position). The connection protector 400 also includes the first cover portion 11, the second cover portion 12 and the living hinge 51. The connection protector 400 comprises two of the tamper-proof locking assembly 60, such as a double latch system. The unlocking mechanism of the double latch system is non-intuitive and requires the action of two hands simultaneously, such as pushing up two latches of the double latch system in the same or opposite directions simultaneously. Each of the tamper-proof locking assemblies 60 in the double latch system in FIGS. 4A-4C can include opposite tongue locking component 603 and groove locking component 604 mirrored on the first cover portion 11 and the second cover portion 12 of the connection protector 400. The double latch system 60 provides a way to reduce accidental disconnections or tampering because it requires two hands to release and open.

In one aspect, the connection protector 400 can have color-coding 95 (shown in FIG. 4A) for safety considerations, akin to color-coded medication labels or color-coded IV tubing. The color-coding 95 can be used to coordinate with relevant medical events or medical treatments, which provides the advantages of easy access to designated medical lines (e.g., the first and second tubes 20 and 21) by reducing human errors.

The connection protector 400 can further include a monitoring system 65 configured to monitor the state (e.g., locked or unlocked) of the tamper-proof locking assembly 60 (e.g., the locking component 603 and the groove locking component 604) and/or the state (e.g., open or closed) of the first and second cover portions 11 and 12 and transmit a signal based on the states. For example, the monitoring system 65 can include a sensor 651 configured to sense whether the locking component 603 and groove locking component 604 are unlocked (e.g., by a patient) and/or the first and second cover portions 11 and 12 are in open position, and a transmitter 652, such as a wireless transmitter, configured to transmit a signal to a receiver, which can be held by a nurse or installed at a nurse station, to alert that the locking component 603 and groove locking component 604 are unlocked and/or the first and second cover portions 11 and 12 are in open position.

The connection protector 400 shown in FIG. 4A is a hollow cylinder. FIG. 4A′ shows that the connection protector 400 is a rectangular box.

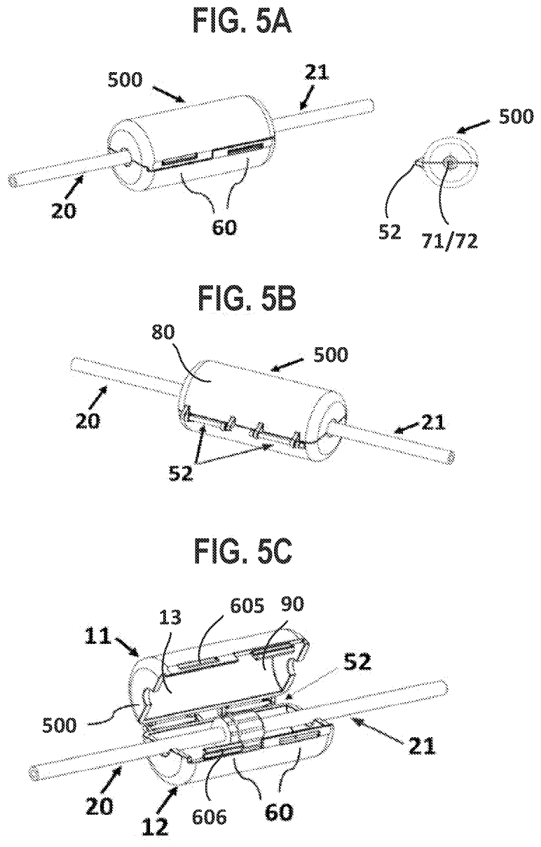

An exemplary embodiment of a connection protector 500 for enclosing a connection point of two conveying devices, such as tubes, lines and drains, is shown in FIG. 5A (perspective view showing the tamper-proof locking assemblies 60 and side view showing the proximal/distal opening 71/72 and a mechanical hinge 52), FIG. 5B (schematic view showing the mechanical hinge 52) and FIG. 5C (open position). The connection protector 500 also includes the first cover portion 11 and the second cover portion 12. The mechanical hinge 52 connects the first cover portion 11 and the second cover portion 12. The connection protector 500 further includes two of the tamper-proof locking assembly 60, such as a double latch system. The unlocking mechanism of the double latch system is non-intuitive and requires the action of two hands simultaneously, such as pushing up two latches of the double latch system in the same or opposite directions simultaneously. Each of the tamper-proof locking assembly 60 of the double latch system in FIGS. 5A and 5C includes a female component 605 and a male component 606 allowing the female and male components 605 and 606 to snap together in locked position. In addition, the mechanical hinge 52 provides a mechanism to open and close the connection protector 500. Mechanical hinging allows a smooth opening and closing of the connection protector, making it easy to open the connection protector 500 completely without any elastic bounce.

In one aspect, the outer portion of the connection protector 500 can include an outer shell (e.g., a soft air-filled outer shell) 80, as shown in FIG. 5B. The soft air-filled outer shell 80 can provide a soft, flexible, or gel-like surface allowing the adherence of the connection protector 500 to the skin of the patient or dressing, such as DuoDERM dressings or Tegaderm dressings.

In one aspect, the connection protector 500 further includes an inner core (or mold) 90 (as shown in FIG. 5C), such as a rigid or hard inner core received in the internal compartment 13 for protecting the connection point 30 and the first and second tubes 20 and 21. For example, the rigid inner core 90 has the structure of providing stability to the first and second tubes 20 and 21. The configuration of the rigid inner core 90 of the connection protector 500 is designed to stabilize the connection point 30 and the first and second tubes 20 and 21. In one aspect, the connection protector 500 has a larger dimension and size to accommodate a larger tube or drain, such as a gastrostomy tube (G-tube) extension. In an embodiment, the inner core 90 can have a rough surface, to impede the movement of the connection point 30 and the first and second tubes 20 and 21 across it.

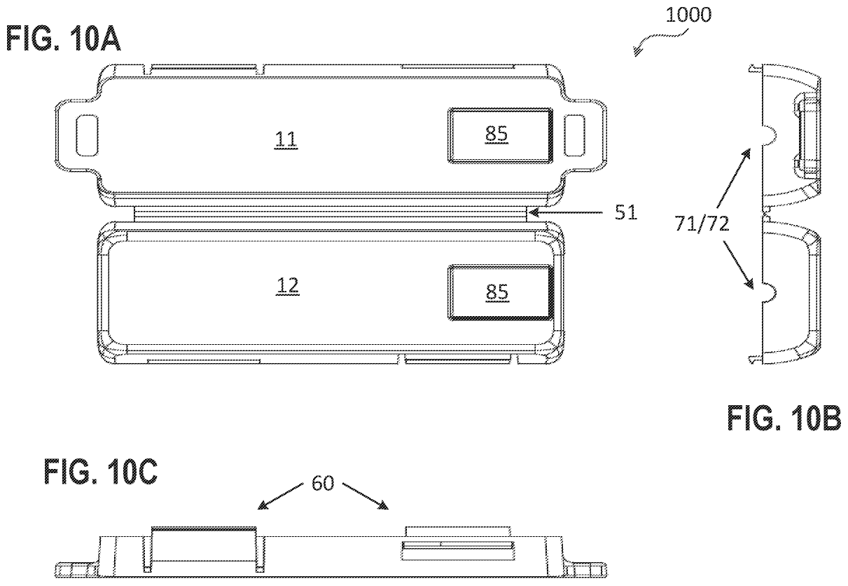

An exemplary embodiment of a connection protector 1000 for enclosing a connection point of two conveying devices, e.g., tubes, lines and drains, is shown in FIG. 10A (top view in open position), FIG. 10B (side view) and FIG. 10C (front view). The connection protector 1000 can also include the first cover portion 11, the second cover portion 12, the living hinge 51, the tamper-proof locking assembly 60, the openings 71/72 and the visual access 85.

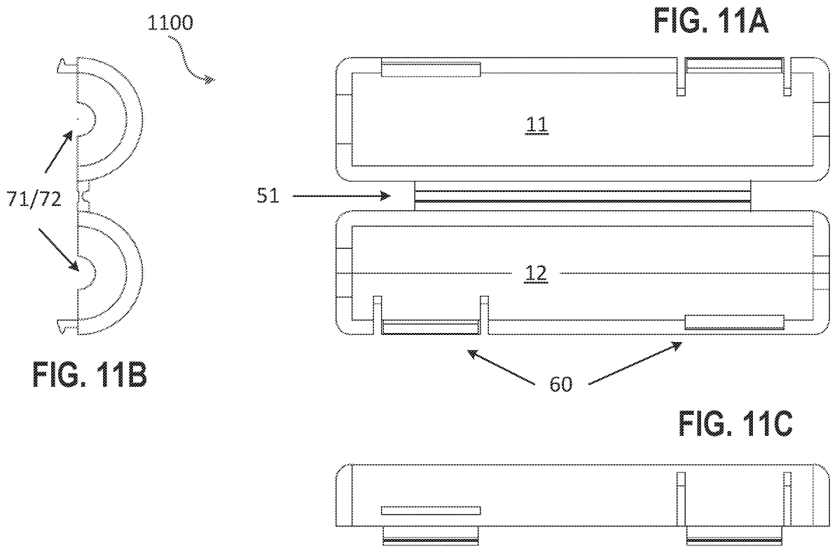

An exemplary embodiment of a connection protector 1100 for enclosing a connection point of two conveying devices, e.g., tubes, lines and drains, is shown in FIG. 11A (top view in open position), FIG. 11B (side view) and FIG. 11C (front view). The connection protector 1100 can also include the first cover portion 11, the second cover portion 12, the living hinge 51, the tamper-proof locking assembly 60 and the openings 71/72.

The configuration of a connection protector according to the present disclosure provides various dimensions to accommodate tubes or lines which have various sizes, shapes, or dimensions. In one aspect, the connection protector has various shapes, such as a hollow cylinder (FIGS. 2 and 4A), a rectangular box (FIGS. 3A and 4A′) or a solid cylinder (FIG. 5C).

In one aspect, an internal portion or a central portion of the internal compartment 13 is configured to hold the connection point 30 of the first and second tubes 20 and 21 together by blocking the back ends of the connected first and second tubes 20 and 21 from pulling apart or being unscrewed. In some other embodiments, some specific features can be added to the designs of the connection protector according to the present disclosure to increase the safety protection or to strengthen the securement of the first and second tubes 20 and 21. For example, a fence closure (e.g., the fence closure 70 with strain relief) can be added to the connection protector (e.g., the connection protector 300 shown in FIG. 3C) to increase the safety protection or to strengthen the securement of the first and second tubes 20 and 21. As another example, the rigid/hard inner core 90 can be added to the internal compartment 13 of the connection protector 500 to protect and stabilize the connection point 30 of the first and second tubes 20 and 21.

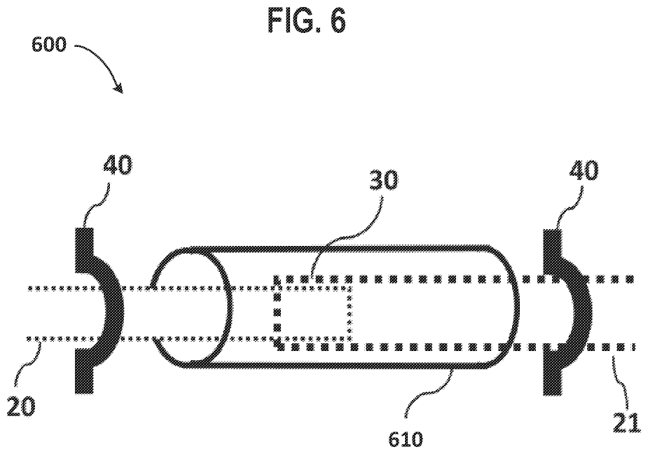

An exemplary embodiment of a securement system 600 for protection a connection point of two conveying devices, such as tubes, lines and drains, is shown in FIG. 6. The securement system 600 can include a connection protector 610, e.g., the connection protector 100/200/300/400/500, and at least one securing patch 40. The connection protector 610 is configured to enclose a connection point of two tubes. For example, the connection protector 610 can includes a first cover portion (e.g., the first cover portion 11), a second cover portion (e.g., the second cover portion 12), an internal compartment (e.g., the internal compartment 13), a hinge (e.g., the hinge 50, the living hinge 51 or the mechanical hinge 52), two openings (e.g., the proximal and distal openings 71 and 72), at least one tamper-proof locking assembly (e.g., the tamper-proof locking assembly 60), a monitoring system (e.g., the monitoring system 65), fence closures with strain relief (e.g., the fence closures 70 with strain relief), an outer shell (e.g., the soft air-filled outer shell 80), a clear visual access (e.g., the visual access 85), an inner core (e.g., the inner core 90), and color-coding (e.g., the color-coding 95). In an embodiment, the securement system 600 can further include a securing patch 40 that is configured to secure the connection protector 610 to an object, such as clothing of patients. In another embodiment, the securement system 600 can further include two securing patches 40 that are to be placed on either side of the proximal and distal openings 71 and 72 of the connection protector 610 to secure the first and second tubes 20 and 21 to an object. In one aspect, the securing patch 40 can have a lock function, such as a grip lock, a stat lock, or a stat lock-like device. In an embodiment, the securing patch 40 can be placed in a location in close proximality of the connection protector 610, such as close to the proximal and distal openings 71 and 72 of the internal compartment 13 of the connection protector 610. The feature of a grip lock includes an adhesive product which can be used to secure lines (e.g., the first and second tubes 20 and 21) to the patient's skin or clothing. The feature of a stat lock includes a device designed to secure vulnerable lines (typically central venous catheters) from being dislodged or disengaged from the patient's skin/insertion site. The feature of the stat lock-like device includes a device on either side of the tamper-proof connection protector 610 which is attached to the lines, tubes, drains, or other medical devices (e.g., the first and second tubes 20 and 21) in order to prevent the connections from coming apart.

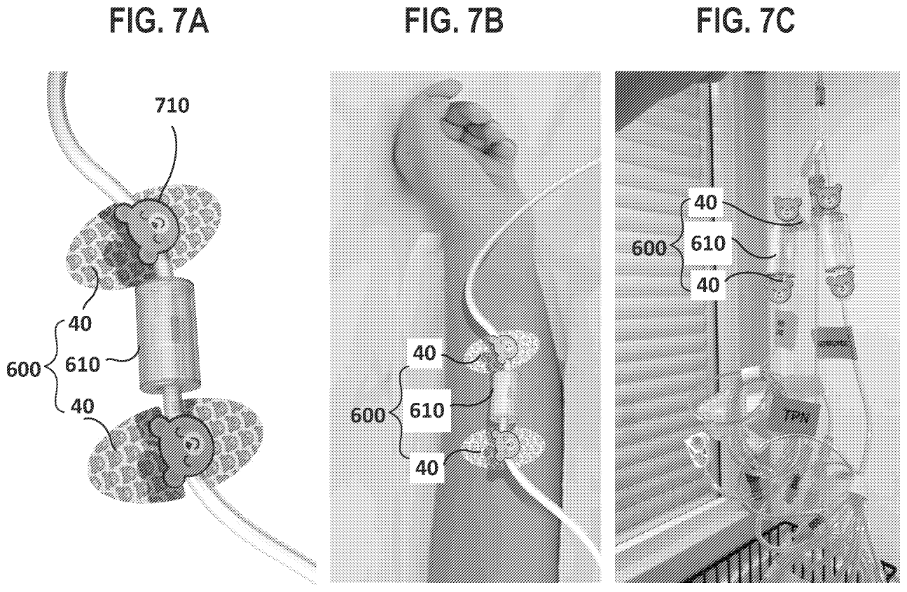

In one aspect, the securing patch 40 can have a cheerful design 710, such as a smiling bear, as shown in FIG. 7A, adding warmth and humor to a potential scary environment with collections of medical equipment. In one aspect, a line or a tube (e.g., the first and second tubes 20 and 21) is inserted into the internal compartment 13 of the connection protector 610, and can fall freely into the internal compartment 13 from either of the proximal and distal openings 71 and 72. In one aspect, the connection protector 610 can be used to secure various connections including chest tube drains, central venous lines, double and triple connectors for CVL's, peripheral IV lines, urine catheter drainage systems, ostomy drainage systems, gastrostomy drainage, duodenostomy drainage, or feeding systems. In one aspect, the connection protector 610 has a box-like design having a cover (e.g., a combination of the first cover portion 11 and the second cover portion 12) and a lock (e.g., the tamper-proof locking assembly 60). The cover can be snapped-shut and opened easily by pressing in to dis-engage the lock. The box can be free moving for protecting the medical lines and connections (e.g., the first and second tubes 20 and 21) without attaching to the patient. The box can also to be connected to the medical lines easily. In one aspect, the box-like connection protector 610 can be connected to a bed or to a patient by using the posey attachment slots at the end of each side of the connection protector for the use of protecting medical lines which are attached to bed-bound or mobile patients.

The connection protector 100/200/300/400/500/610 and/or the securement system 600 according to the present disclosure provide unique designs of protecting the connection points of two tubes (or lines, drains, etc.) from avoiding the force of pulling on the tubes, which provides the ability of preventing the unscrewing and disconnecting of the tubes completely. The connection protector 100/200/300/400/500/610 and/or securement system 600 according to the present disclosure can also be used to secure the tubes to the patient with stat lock technology, or freely hanging. The prevention of tube disconnection can save countless amounts of patients and families from undue stress, illness and injury. In addition, the designs and configurations of the connection protector 100/200/300/400/500/610 and/or securement system 600 can be manufacture cost-effectively, such as 3D printing, to offer an advantage of affordability, which are readily available for private and public industries.

The features and properties of the connection protector 100/200/300/400/500/610 and the securement system 600 are shown in examples 1-3, which illustrate the benefits and advantages of the present disclosure.

### Example 1: The Use of the Securement System 600 to Protect a Connection Point of Medical Lines (e.g., the Connection Point 30 of the First and Second Tubes 20 and 21)

The securement system 600 including the tamper-proof connection protector 610 and two securing patches 40 is used to protect a connection point of medical lines (e.g., the connection point 30 of the first and second tubes 20 and 21). FIG. 7A shows the configuration of the connection protector 610 in closed position enclosing the connection point of the medical lines or tubes. In one example, the securing patch 40 has a lock function, such as a grip lock, a stat lock, or a stat lock-like device, and is placed in a location in close proximality of the connection protector 610, such as close to the openings 71 and 72 of the internal compartment 13 of the connection protector 610. In one example, the outer portion of the connection protector 610 includes an outer shell (e.g., the soft air-filled outer shell 80) which allows the adherence of the connection protector 610 to the skin of the patient or dressing. FIG. 7A shows the use of the securement system 600 in combination of two dressings, such as DuoDERM dressings or Tegaderm dressings. The configuration of the connection protector 610 provides various dimensions to accommodate tubes or lines (e.g., the first and second tubes 20 and 21) which have various sizes, shapes, or dimensions. In one example, the connection protector 610 has a larger size for enclosing a connection point of drainage tubes. In another example, the connection protector 610 has an inner core (e.g., the rigid inner core 90) for protecting the connection point 30 and the first and second tubes 20 and 21. In an embodiment, the rigid inner core 90 has the structure of providing stability to the connected first and second tubes 20 and 21. The configuration of the rigid inner core 90 of the connection protector 610 is designed to stabilize the connected first and second tubes 20 and 21.

### Example 2: The Use of the Connection Protector 100/200/300/400/500/610 and/or Securement System 600 to Protect Medical Lines which are Attached to Patients' Bodies

The connection protector 100/200/300/400/500/610 and/or securement system 600 according to the present disclosure are used to protect medical lines which are attached directly or indirectly to the body of the patient. FIGS. 7B and 7C show the uses of multiple securement systems (e.g., the securement systems 600) for protecting the connection points of medical lines or tubes, wherein each of the securement systems 600 has a connection protector (e.g., the connection protector 610) and two securing patches (e.g., the securing patches 40). The connection protector 610 which is tamper-proof is used to enclose the connection point of medical lines (e.g., the connection point 30 of the first and second tubes 20 and 21) which were attached directly (as shown in FIG. 7B) or indirectly (as shown in FIG. 7C) to the body of the patient using a secondary securement system, sleeve (e.g., CP sleeve), holding device, etc. that secures a combination of the connection protectors to the patient's clothing or to the bed of a stable, immobile patient. In one example, the securement system 600 is used in combination of two dressings, such as DuoDERM dressings or Tegaderm dressings. In one example, the outer portion of the connection protector 610 includes an outer shell, e.g., the soft air-filled outer shell 80, which provides a soft, flexible, or gel-like surface allowing the adherence of the connection protector 610 to the skin of the patient as shown in FIG. 7B. In one example, the connection protector 610 had an inner core, e.g., the rigid/hard inner core 90, to protect and stabilize the connection point and the tubes.

### Example 3: The Use of the Connection Protector 100/200/300/400/500/610 and/or Securement System 600 to Protect the Connection Point of a Drainage System 800

The connection protector 100/200/300/400/500/610 and/or the securement system 600 according to the present disclosure are used to secure and protect a connection point of a drainage system 800. The drainage system 800 includes a drainage bag 810, a collection tube 820 which provides an outlet/inlet or opening of the drainage bag 810, and a Gastrostomy-tube (G-tube) extension (or port) 830. The connection protector 610 for protecting the drainage system 800 has a specific design (e.g., having the rigid/hard inner core 80) which is configured to enclose a connection point 840 of the collection tube 820 and the G-tube extension 830 (e.g., a G-tube port). In particular, the connection protector 610 has a tamper-proof design (i.e., a connection protector including at least one tamper-prof locking assembly) and a larger size or dimension to accommodate the protruding arm of the G-tube extension 830. In one example, the connection protector 610 has a visual access (e.g., the visual access 85) which is clear or transparent to provide the advantage of allowing the medical staff to quickly verify the connection point 840 without opening the connection protector.

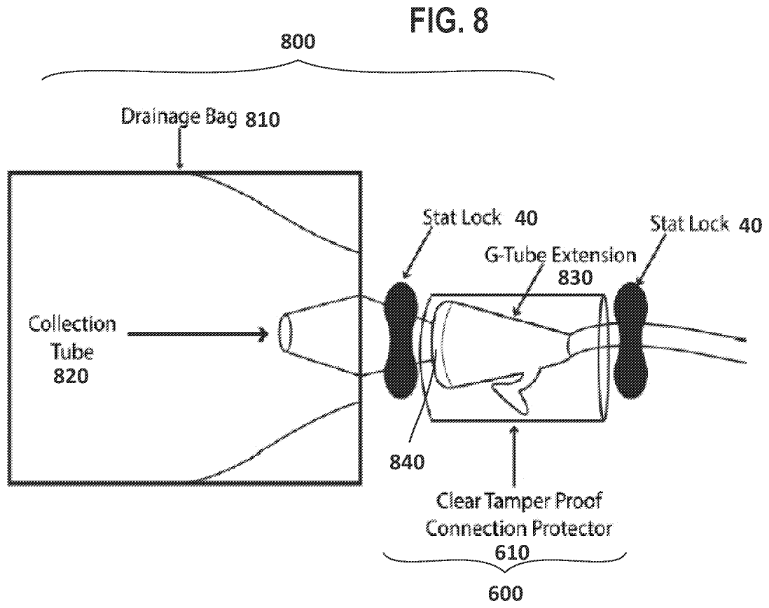

The securement system 600 shown in FIG. 8 includes a connection protector (e.g., the connection protector 610) and two securing patches (e.g., the securing patches 40). In an embodiment, the securing patch 40 has a lock function, such as a grip lock, a stat lock, or a stat lock-like device. The securing patch 40 can be placed in a location in close proximality of the connection protector 610, such as close to the proximity and distal openings 71 and 72 of the internal compartment 13 of the connection protector 610. In one example, the securement system 600 is used in combination of two dressings, such as DuoDERM dressings, medical foam or Tegaderm dressings. It should be noted that the skin under or surrounding any device should be assessed on a regular basis per individual hospital policy. In one example, the outer portion of the connection protector 610 includes an outer shell (e.g., the soft air-filled outer shell 90) which provides a soft, flexible, or gel-like surface allowing the adherence of the connection protector 610 to the skin of the patient. In one example, the connection protector 610 had an inner core (e.g., the rigid/hard inner core 80) to protect and stabilize the connection point and the tubes.

In addition, the connection protector 100/200/300/400/500/610 and/or securement system 600 according to the present disclosure have various sizes, such as larger, wider, or longer, which can be used to secure and protect a connection point of a variety of drainage systems having various sizes and/or shape. These types of drainage systems have unique shapes or sizes which can be disconnected more easily. The connection protector 100/200/300/400/500/610 and/or securement systems 600 according to the present disclosure can provide adequate protection to prevent the disconnection of the connection points. The example of the drainage system includes but is not limited to: (1) connecting a G-tube extension and a gravity drainage tube, (2) connecting a feeding extension tubing and a G-tube, (3) connecting a kangaroo feeding set and an extension feeding tubing, (4) connecting an alternate drainage tube and a drainage bag, and (5) connecting a drainage bag to a G-tube and a duodenostomy bag.

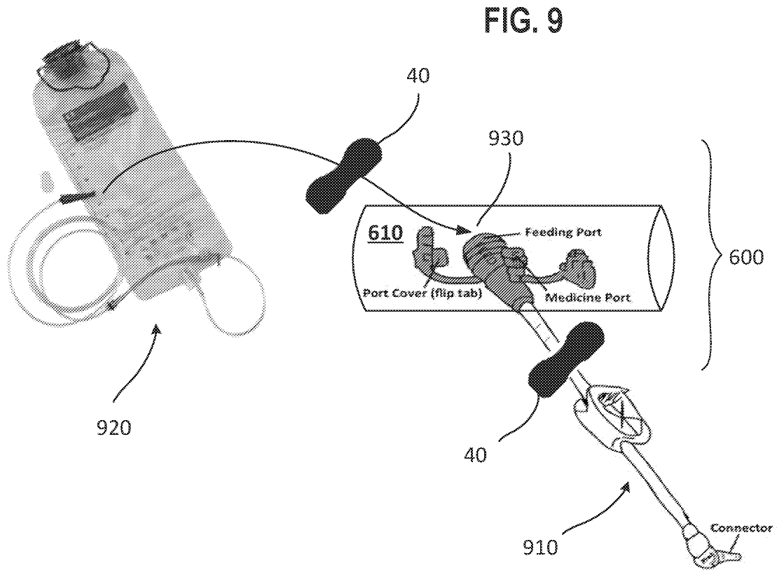

FIG. 9 illustrates the use of the connection protector 610 and/or securement system 600 to protect a connection point 930 of an extension feeding tubing 910 and a kangaroo feeding set 920. The securement system 600 includes two securing patches 40 for securing the extension feeding tubing 910 and the kangaroo feeding set 920, respectively.

It is to be understood that the present disclosure is not to be limited to the exact description and embodiments as illustrated and described herein. To those of ordinary skill in the art, one or more variations and modifications will be understood to be contemplated from the present disclosure. Accordingly, all expedient modifications readily attainable by one of ordinary skill in the art from the disclosure set forth herein, or by routine experimentation therefrom. This device may be modified, made of a material more compatible for veterinary use, i.e., stainless steel, indestructible aluminum, etc.

What is claimed is:

1. A medical connection protector for protecting a connection point of a first conveying device and a second conveying device, comprising:
a first cover portion and a second cover portion, a combination of which forms an internal compartment configured to enclose a connection point of a first conveying device and a second conveying device;
a hinge coupled to a first side of each of the first and second cover portions, the hinge configured to enable the first cover portion and the second cover portion to be in an open or closed position,
two openings formed on both ends of the first and second cover portions, allowing the first and second conveying devices to pass respectively therethrough; and
a first tamper-proof locking assembly attached to a second side of each of the first and second cover portions, the first tamper-proof locking assembly configured to lock the first cover portion to the second cover portion.

2. The medical connection protector of claim 1, wherein the first tamper-proof locking assembly includes a first female component attached to the second side of the first cover portion and a first male component attached to the second side of the second cover portion, the first female component and the first male component being configured to snap together in locked position.

3. The medical connection protector of claim 2, further comprising a second tamper-proof locking assembly that includes a second female component attached to the second side of the first cover portion and a second male component attached to the second side of the second cover portion, the second female component and the second male component configured to snap together in locked position.

4. The medical connection protector of claim 2, further comprising a second tamper-proof locking assembly that includes a second female component attached to the second side of the second cover portion and a second male component attached to the second side of the first cover portion, the second female component and the second male component configured to snap together in locked position.

5. The medical connection protector of claim 1, wherein the first tamper-proof locking assembly has a safety-lock, an engageable lock, or a releasable lock.

6. The medical connection protector of claim 1, wherein the hinge is a living hinge integrated with the first sides of the first and second cover portions.

7. The medical connection protector of claim 1, wherein the hinge is a mechanical hinge attached to the first sides of the first and second cover portions.

8. The medical connection protector of claim 1, wherein one of the openings has a same diameter as an outer diameter of the first conveying device.

9. The medical connection protector of claim 1, further comprising color coding provided on the first cover portion indicating a type of conveying device.

10. The medical connection protector of claim 1, further comprising an inner core received in the internal compartment, the inner core configured to accommodate the connection point of the first and second conveying devices.

11. The medical connection protector of claim 10, wherein the inner core has a same configuration of a shape of the connection point of the first and second conveying devices.

12. The medical connection protector of claim 10, wherein the inner core is a rigid inner core and/or has a rough surface.

13. The medical connection protector of claim 1, further comprising a monitoring system configured to monitor a state of the tamper-proof locking assembly and/or the first and second cover portions and transmit a signal based on the state.

14. The medical connection protector of claim 1, further comprising a fence closure disposed in the internal compartment, the fence closure configured to enclose the first conveying device and prevent the first conveying device from disconnection with the second conveying device.

15. The medical connection protector of claim 14, wherein the fence closure includes two walls installed on the first and second cover portions, respectively, the two walls overlapping when the first and second cover portions is in the closed position.

16. The medical connection protector of claim 1, further comprising a visual access formed on the first cover portion, allowing the connection point of the first and second conveying devices to be visually accessed from external the medical connection protector.

17. The medical connection protector of claim 1, wherein the first cover portion is made of a transparent material.

18. The medical connection protector of claim 1, wherein the medical connection protector is made of a biocompatible and/or antimicrobial material.

19. A medical securement system comprising the medical connection protector of claim 1 and at least one securing patch, the at least one securing patch configured to secure the medical connection protector and/or the first conveying device to an object.

20. The medical securement system of claim 19, wherein the securing patch is a grip lock or a stat lock.
